(19) United States
(12) Patent Application Publication
(10) Pub. No.: US 2026/0325671 A1
(43) Pub. Date: Oct. 22, 2026

(54) YEAST PLATFORM

(71) Applicant: Fzata, Inc., Halethorpe, MD (US)

(72) Inventor: Hua Yu, Columbia, MD (US)

(73) Assignee: Fzata, Inc., Halethorpe, MD (US)

(21) Appl. No.: 19/485,205

(22) PCT Filed: May 17, 2024
(86) PCT No.: PCT/US2024/030115

§ 371 Date: Nov. 17, 2025

Related U.S. Application Data
(60) Provisional application No. 63/467,484, filed on May 18, 2023.

(51) Int. Cl. A61K 48/00; C07K 14/52; C07K 14/54; C07K 16/1282; C12N 1/16; C12N 9/04; C12R 1/85

(52) U.S. Cl. CPC A61K 48/0008; C07K 14/52; C07K 14/54; C07K 16/1282; C12N 1/16; C12N 9/0006; C12Y 101/01085; C12R 2001/85

(57) ABSTRACT

The present disclosure is in the field of bioengineered yeast strains as a therapeutic platform. In particular, engineered strains of Saccharomyces boulardii yeast comprising a nucleic acid encoding a modified 3-isopropylmalate dehydrogenase (Leu2) and a therapeutic protein and methods for treating or preventing a disease or conditions in a subject in need.

### REFERENCE TO RELATED APPLICATIONS

This application claims the benefit of the priority date of U.S. provisional application, 63/467,484, filed May 18, 2023, the contents of which are incorporated herein by reference in their entirety.

### GOVERNMENT SUPPORT

The invention was made with government support under R44AI155277 and R44DK129133 awarded by the National Institutes of Health. The government has certain rights in the invention.

### TECHNICAL FIELD

The present disclosure is in the field of bioengineered yeast strains as a therapeutic platform. Described are engineered strains of Saccharomyces boulardii yeast comprising a nucleic acid encoding a modified 3-isopropylmalate dehydrogenase (Leu2) and a therapeutic protein and methods for treating or preventing a disease or conditions in a subject in need thereof.

### BACKGROUND

The following discussion is merely provided to aid the reader in understanding the disclosure and is not admitted to describe or constitute prior art thereto.

Therapy using biologic protein based drugs is currently hampered by issues such as needing to be injected or intravenously infused and accordingly, there is currently a need for patient friendly, orally administered protein therapies. Engineered strains of Saccharomyces boulardii (S. boulardii) yeast for oral administration have presented as a promising therapeutic option. However, producing an engineered S. boulardii therapeutic platform to produce any desired therapeutic protein in order to treat a particular disease or condition has been challenging due to genetic instability and low transgene copy number.

Using a modified 3-isopropylmalate dehydrogenase (Leu2) selection transgene expression cassette has shown to result in increased genetic stability of the selected transgene and increased constitutive expression of the therapeutic protein over generations spanning the manufacturing process, making the resultant engineered S. boulardii a desirable platform for “plug and play” gene therapeutics.

### SUMMARY

In one aspect, the present disclosure provides, an engineered strain of Saccharomyces boulardii yeast comprising a nucleic acid encoding 3-isopropylmalate dehydrogenase (Leu2) and at least one site-specific chromosomal insertion of a nucleic acid (i.e., a transgene) encoding a therapeutic protein.

In some embodiments, the nucleic acid encoding Leu2 comprises one or more modifications selected for a mutation or deletion.

In some embodiments, the one or more modifications reduce enzymatic activity of Leu2.

In some embodiments, the Leu2 comprises an amino acid sequence selected from SEQ ID NOs: 2 or 3.

In some embodiments, the yeast comprises a complete or partial deletion of wild-type Leu2.

In some embodiments, the engineered strain of Saccharomyces boulardii yeast comprises complete or partial deletion of URA3.

In some embodiments, the engineered strain of Saccharomyces boulardii yeast complete or partial deletion of GAP1.

In some embodiments, the therapeutic protein is selected from a binding protein, an antigen-binding domain, an immunoglobulin, an antibody, a cytokine, a glycoprotein, an anti-cytokine, a hormone, a chemokine, an enzyme, an anti-microbial peptide, or any combination thereof.

In some embodiments, the nucleic acid encoding the therapeutic protein is incorporated into at least two different chromosomes.

In some embodiments, the engineered strain of Saccharomyces boulardii yeast comprises a nucleic acid encoding a Leu2 promoter.

In some embodiments, the Leu2 promoter comprises a nucleic acid sequence selected from SEQ ID NOs: 4 or 5.

In some embodiments, the therapeutic protein is the binding protein comprising a structure selected from VHH, Fc-VHH, VHH-Fc, VHH-VHH, VHH-VHH-VHH-VHH, Fc-VHH-VHH, VHH-Fc-VHH, and VHH-VHH-Fc, wherein each or any of the VHH or Fc domains are attached to another VHH or Fc domain via an optional linker sequence.

In some embodiments, the therapeutic protein is a binding protein that binds to TcdA, TcdB, or a combination thereof.

In some embodiments, the therapeutic protein comprises an amino acid sequence of SEQ ID NO: 6.

In some embodiments, the therapeutic protein is a binding protein that binds to TNF-α.

In some embodiments, the binding protein is an IgG or comprises at least one VHH domain.

In some embodiments, the therapeutic protein comprises an amino acid sequence of SEQ ID NOs: 7 or 8.

In some embodiments, the therapeutic protein is a binding protein that binds to TNF-α and IL-17A.

In some embodiments, the binding protein is an IgG or comprises at least two VHH domains.

In some embodiments, the therapeutic protein comprises an amino acid sequence of SEQ ID NOs: 9-11.

In some embodiments, the therapeutic protein expresses human cytokine IL-10.

In some embodiments, the IL-10 is Fc-fused or non Fc-fused.

In some embodiments, the therapeutic protein comprises an amino acid sequence of SEQ ID NOs: 15.

In some embodiments, the therapeutic protein expresses human cytokine IL-22.

In some embodiments, the IL-22 is Fc-fused or non Fc-fused.

In some embodiments, the therapeutic protein comprises an amino acid sequence of SEQ ID NOs: 16.

In some embodiments, the therapeutic protein expresses human hormone insulin.

In some embodiments, the insulin is Fc-fused or non Fc-fused.

In some embodiments, the therapeutic protein comprises an amino acid sequence of SEQ ID NOs: 17.

In some embodiments, the engineered strain of Saccharomyces boulardii yeast comprises at least 2, at least 3, at least 4, at least 5, at least 6, at least 7, at least 8, at least 9, or at least 10 or more copies of the nucleic acid encoding the therapeutic protein incorporated into the yeast genome.

In some aspect, the present disclosure provides, a method of treating or preventing a disease or condition comprising orally administering to a subject in need thereof a therapeutically effective amount of the engineered strain of Saccharomyces boulardii yeast.

In some embodiments, the disease or condition is selected from an inflammatory condition, an infection, irritable bowel syndrome (IBS), malignancy, neurodegenerative disease, diabetes, obesity, fatty liver disease, metabolic disease, graft-versus-host disease (GVHD), an autoimmune disease, or pain.

In some embodiments, the inflammatory condition is selected from inflammatory bowel disease (IBD), gut inflammation, Crohn's disease, and ulcerative colitis.

In some embodiments, the infection is a C. difficile infection.

In another aspect, the present disclosure provides, a method of expressing a therapeutic protein is a subject, comprising orally administering to the subject the engineered strain of Saccharomyces boulardii yeast.

In one aspect, the present disclosure provides, a pharmaceutical composition comprising an engineered strain of Saccharomyces boulardii yeast and a pharmaceutically acceptable carrier or diluent.

In some aspects, the present disclosure provides for a use of an engineered strain of Saccharomyces boulardii yeast of any one of claims 1-30 in the manufacture of a medicament for the treatment of a disease or condition, wherein the disease or condition is selected from an inflammatory condition, an infection, irritable bowel syndrome (IBS), neurodegenerative disease, diabetes, obesity, fatty liver disease, malignancy, metabolic disease, graft-versus-host disease (GVHD), an autoimmune disease, or pain.

In one aspect, the present disclosure provides for a use of an engineered stain of Saccharomyces boulardii yeast for expressing a therapeutic protein is a subject.

Both the foregoing summary and the following description of the drawings and detailed description are exemplary and explanatory. They are intended to provide further details of the disclosure, but are not to be construed as limiting. Other objects, advantages, and novel features will be readily apparent to those skilled in the art from the following detailed description of the disclosure.

It should be appreciated that all combinations of the foregoing concepts and additional concepts discussed in greater detail below are provided as being part of the inventive subject matter disclosed herein and may be employed in any combination to achieve the benefits described herein.

### BRIEF DESCRIPTION OF THE DRAWINGS

FIG. 1 depicts expression levels of clones with DHFR and modified Leu2 selection in individual screened S. boulardii clones transformed with exemplary Leu2 selection cassette or DHFR selection cassette. Expression levels of therapeutic protein were measured by ELISA assays. YPD cultured supernatants were diluted 40-fold for 02 DHFR clones and 40-fold for 02_Leu2 clones (FIG. 1A), 25-fold for 06_DHFR and 40-fold for 06 Leu2 clones (FIG. 1B) and 5-fold for 06m_DHFR clones and 10-fold for 06m_Leu2 clones (FIG. 1C).

FIG. 2 depicts expression levels of antibody against cytokines in engineered yeast cells transformed with an antibody-encoding therapeutic transgene within a modified Leu2 cassette (Leu2 strain) or DHFR cassette (DHFR strain). Strains (06_Leu2 and 06_DHFR) were incubated in YPD for 3 hours to test acute expression of the antibody ((anti-TNF-α (human)) from Leu2 and DHFR-selection strains (FIG. 2A). Also, the same strains from FIG. 2A were incubated in YPD for 24 h to test the antibody expression level (FIG. 2B). FZY085 strains are engineered yeast cells expressing antibody against TNF-α (mouse). The antibody expression level was determined by ELISA of a serials of diluted supernatant from a culture of 3 hours' incubation in YPD, with each dilution as represented on the x axis (FIG. 2C; circles represent the Leu2 strain and squares represent the DHFR strain). The supernatant was serially diluted 2-fold at dilution factor 2, 4, 8, 16, 32, 64, 128, 256, 512, and 1024. The same strains were also incubated in YPD for 24 hours to test 24 hour expression of Leu2 strain and DHFR strain and supernatant was serially diluted 2-fold at dilution factor 10, 20, 40, 80, 160, 320, 640, 1280, 2560, and 5120. The antibody expression level was determined by ELISA in each dilution as represented on the x axis (FIG. 2D; circles represent the Leu2 strain and squares represent the DHFR strain)

FIG. 3 depicts 3 hour acute expression and 24 hour expression of an exemplary therapeutic protein determined by ELISA in engineered yeast cells transformed with a therapeutic transgene in a modified Leu2 GOI cassette (Leu2 strains 02_3 and 02_4). The engineered yeast cells were cultured in M2 (synthetic media with Leu2 selection plus yeast extracts) medium for 20, 44, 52, or 112 hours as depicted on the x axis followed by 3 hours of culture in YPD medium (left) or 24 hours (right).

FIG. 4 depicts copy number of an exemplary therapeutic transgene in engineered yeast cells transformed with a therapeutic transgene in a modified Leu2 GOI cassette (Leu2 strains 02_3 and 02_4) and DHFR GOI cassette (DHFR strains 02_1 and 02_2). DHFR strains were cultured in M3 (M2 media supplemented with Leucine) medium and passage daily for 10 days. Copy number of the therapeutic transgene was determined from harvested genomic DNA at passage day 0, day 2, and day 10 at site 1 and site 2 (FIG. 4A). Leu2 strains were cultured in M1 (Synthetic media with Leu2 selection) medium and passage daily for 10 days and in M2 medium passaged daily for about 5 days (112 h). Copy number of the therapeutic transgene was determined from harvested genomic DNA at passage day 0, day 2, and day 10 at site 1 and 2 for Leu2 strain 02_3 and site 1 for 02_4 (FIGS. 4B and 4C).

FIG. 5 depicts the percentage of engineered yeast cells expressing an exemplary therapeutic transgene following transformation with the therapeutic transgene in a modified Leu2 GOI cassette (Leu2 strains 02_3 and 02_4) and DHFR GOI cassette (DHFR strains 02_1 and 02_2) during a continuous long-term culture (10 passages). Leu2 strains were cultured in M2 medium or M1 medium for 10 days and DHFR strains were cultured in M3 medium for 10 days. Cultured cells were spread on corresponding plate at day 0 (P0) and day 10 (P10) and single colonies grown on these plate were randomly selected and seeded into YPD medium in a 96-well culture plate for an overnight shaking culture. The therapeutic protein expression was detected by ELISA using the culture supernatant from each single colony. Clones with positive expression of therapeutic protein are identified as specific protein-expressing cells.

FIG. 6 depicts growth of engineered yeast cells expressing an exemplary therapeutic transgene after transformation with the therapeutic transgene in a modified Leu2 GOI cassette (Leu2 strains 02_3 and 02_4) and DHFR GOI cassette (DHFR strains 02_1 and 02_2). Leu2 strains were cultured in M2 medium or M1 medium for 10 days and DHFR strains were cultured in M3 medium for 10 days with daily passage. OD600 was measured to evaluate growth rates at day 1 (P1), day 3 (P3), day 5 (P5), day 8 (P8) and day 10 (P10) (FIG. 6A). Leu2 strains 02_3 and 02_4 were cultured in M2 medium and OD600 was measured at time points across a 120 hour period as depicted on the x axis of FIG. 6B.

FIG. 7 depicts the representative therapeutic protein expression of engineered yeast cells expressing an exemplary therapeutic transgene after transformation with the therapeutic transgene in a modified Leu2 GOI cassette (Leu2 strains 02_3 and 02_4) and DHFR GOI cassette (DHFR strains 02_1 and 02_2). Leu2 strains were cultured in M2 medium or M1 medium and DHFR strains were cultured in M3 medium. Supernatants were assayed for transgene expression by ELISA at Day 0 (P0) and Day 10 (P10) at the different culture medium conditions (FIG. 7A, B).

FIG. 8 depicts the growth rate of engineered yeast cells expressing an exemplary therapeutic transgene after transformation with the therapeutic transgene in a modified Leu2 GOI cassette (Leu2 strains 02_3 and 02_4) and DHFR GOI cassette (DHFR strains 02_1 and 02_2), and parental strains MYA-796, FZY025, and FZY071, and culture in M2 (FIG. 8A) and M1 medium (FIG. 8B).

### DETAILED DESCRIPTION

Embodiments according to the present disclosure will be described more fully hereinafter. Aspects of the disclosure may, however, be embodied in different forms and should not be construed as limited to the embodiments set forth herein. Rather, these embodiments are provided so that this disclosure will be thorough and complete, and will fully convey the scope of the invention to those skilled in the art. The terminology used in the description herein is for the purpose of describing particular embodiments only and is not intended to be limiting.

Unless the context indicates otherwise, it is specifically intended that the various features described herein can be used in any combination. Moreover, the disclosure also contemplates that in some embodiments, any feature or combination of features set forth herein can be excluded or omitted. To illustrate, if the specification states that a complex comprises components A, B, and C (or A, B, and/or C), it is specifically intended that any of A, B or C, or a combination thereof, can be omitted and disclaimed singularly or in any combination.

Unless explicitly indicated otherwise, all specified embodiments, features, and terms intend to include both the recited embodiment, feature, or term and biological equivalents thereof.

### I. Definitions

As used in the description of the invention and the appended claims, the singular forms “a,” “an” and “the” are intended to include the plural forms as well, unless the context clearly indicates otherwise.

The terms “substantially” and “about” are used herein to describe and account for small variations. When used in conjunction with an event or circumstance, the terms can refer to instances in which the event or circumstance occurs precisely as well as instances in which the event or circumstance occurs to a close approximation. When used in conjunction with a numerical value, the terms can refer to a range of variation of less than or equal to ±10% of that numerical value, such as less than or equal to ±5%, less than or equal to ±4%, less than or equal to ±3%, less than or equal to ±2%, less than or equal to #1%, less than or equal to ±0.5%, less than or equal to ±0.1%, or less than or equal to ±0.05%. When referring to a first numerical value as “substantially” or “about” the same as a second numerical value, the terms can refer to the first numerical value being within a range of variation of less than or equal to ±10% of the second numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%. The terms or “acceptable,” “effective,” or “sufficient” when used to describe the selection of any components, ranges, dose forms, etc. disclosed herein intend that said component, range, dose form, etc. is suitable for the disclosed purpose.

Additionally, amounts, ratios, and other numerical values are sometimes presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth.

Also as used herein, “and/or” refers to and encompasses any and all possible combinations of one or more of the associated listed items, as well as the lack of combinations when interpreted in the alternative (“or”).

As used herein, the term “comprising” is intended to mean that the compositions and methods include the recited elements, but not excluding others. “Consisting essentially of” when used to define compositions and methods, shall mean excluding other elements of any essential significance to the composition or method. “Consisting of” shall mean excluding more than trace elements of other ingredients for claimed compositions and substantial method steps. Examples and implementations defined by each of these transition terms are within the scope of this disclosure. Accordingly, it is intended that the methods and compositions can include additional steps and components (comprising) or alternatively including steps and compositions of no significance (consisting essentially of) or alternatively, intending only the stated method steps or compositions (consisting of).

As used herein, “optional” or “optionally” means that the subsequently described event or circumstance can or cannot occur, and that the description includes instances where the event or circumstance occurs and instances where it does not.

The term “antibody” refers to immunoglobulins or immunoglobulin-like molecules including IgA, IgD, IgE, IgG and IgM, combinations thereof or fragments thereof. Fragments of antibodies may include, for example, Fab fragments and single chain variable fragments (scFv). An antibody generally comprises heavy (H) chains and light (L) chains interconnected by disulfide bonds. There are two types of light chain, lambda (λ) and kappa (κ). There are five main heavy chain classes (or isotypes) which determine the functional activity of an antibody molecule: IgM, IgD, IgG, IgA and IgE. Each heavy and light chain contains a constant region and a variable region (also known as “domains”). In combination, the heavy and the light chain variable regions, also called the “Fab region,” specifically bind to a given antigen. Light and heavy chain variable regions contain a “framework” region interrupted by three hypervariable regions, also called “complementarity-determining regions” or “CDRs.” The extent of the framework region and CDRs has been defined (see Kabat et al., Sequences of Proteins of Immunological Interest, U.S. Department of Health and Human Services, 1991). The Kabat database is now maintained online. The sequences of the framework regions of different light or heavy chains are relatively conserved within a species, and framework regions act to form a scaffold that provides for positioning the CDRs in correct orientation by inter-chain, non-covalent interactions.

The CDRs are primarily responsible for binding to an epitope on an antigen. The CDRs of each chain are typically referred to as CDR1, CDR2, and CDR3, numbered sequentially starting from the N-terminus, and are also typically identified by the chain in which the particular CDR is located. Thus, a HCDR3 is located in the variable domain of the heavy chain of the antibody in which it is found, whereas a LCDR1 is the CDR1 from the variable domain of the light chain of the antibody in which it is found. An antibody that binds IL-31RA will have a specific V_{H} region and the V_{L} region sequence, and thus specific CDR sequences. Antibodies with different specificities generally have different CDRs. Although it is the CDRs that vary from antibody to antibody, only a limited number of amino acid positions within the CDRs are directly involved in antigen binding. These positions within the CDRs are called specificity determining residues (SDRs).

The Fc fragment region (Fc) of an antibody plays a role in modulating immune cell activity. The Fc region functions to guarantee that each antibody generates an appropriate immune response for a given antigen, by binding to a specific class of proteins found on certain cells, such as B lymphocytes, follicular dendritic cells, natural killer cells, macrophages, neutrophils, etc. and are called “Fc receptors.” Because the constant domains of the heavy chains make up the Fc region of an antibody, the classes of heavy chain in antibodies determine their class effects. The heavy chains in antibodies include alpha, gamma, delta, epsilon, and mu, and correlate to the antibody's isotypes IgA, IgG, IgD, IgE, and IgM, respectively. Thus, different isotypes of antibodies have different class effects due to their different Fc regions binding and activating different types of receptors.

There are four subclasses of IgG, which is the most abundant antibody isotype found in human serum. The four subclasses, IgG1, IgG2, IgG3, and IgG4, which are highly conserved. The amino acid sequence of the constant regions of these peptides are known in the art, e.g., see Rutishauser, U. et al. (1968) “Amino acid sequence of the Fc region of a human gamma G-immunoglobulin” PNAS 61(4):1414-1421; Shinoda et al. (1981) “Complete amino acid sequence of the Fc region of a human delta chain” PNAS 78(2):785-789; and Robinson et al. (1980) “Complete amino acid sequence of a mouse immunoglobulin alpha chain (MOPC 511)” PNAS 77(8):4909-4913.

As used herein, the terms “antigen-binding domain,” “antigen-binding fragment,” or “antigen-binding protein” refers to antigen binding molecules and fragments that retain the ability to bind to an antigen. Non-limiting examples include single-domain antibodies such as a VHH antibody or a “camelid-like antibody”, and heavy chain only antibodies.

As used herein, the terms “single-domain antibody” or “sdAb” are antibody fragments that have a single monomeric variable antibody domain, which can still bind selectively to a specific antigen.

South American camelids (e.g. alpaca and llama) produce two isotypes of immunoglobulins, IgG2 and IgG3 that lack light chains and are heavy chain antibodies or HCAbs. The VH domain of HCAbs, called VHH fragments, are a single-domain antibody.

The terms “individual,” “subject,” and “patient” are used interchangeably herein, and refer to an individual organism, vertebrate, mammal, or a human. In some embodiments, the individual, subject, or patient is a human.

The terms “treatment” or “treating” as used herein refer to reducing or eliminating a disease or condition and/or improving or ameliorating one or more symptoms of the disease or condition.

The terms “prevent” or “preventing” as used herein refer to precluding a disease or condition from developing in a subject at risk of developing the disease or condition, or precluding the disease or condition from recurrence (i.e., relapse post treatment).

The term “pharmaceutical composition” refers to the combination of at least one active agent with a carrier, inert or active, making the composition especially suitable for diagnostic or therapeutic use in vivo or ex vivo.

The term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see, for example, Martin, Remington's Pharmaceutical Sciences, 15^{th} Ed., Mack Publ. Co., Easton, PA (1975).

### II. Engineered Strain of Saccharomyces boulardii Yeast

Saccharomyces boulardii (S. boulardii) is closely related to Saccharomyces cerevisiae (S. cerevisiae). However, the strains are fundamentally different in terms of ploidy. S. cerevisiae can sporulate and exist in both haploid or diploid states whereas S. boulardii is an obligate diploid that does not sporulate. Due to the diploid status of S. boulardii, it is challenging to genetically engineer S. boulardii with transgenes because of low efficiency of genomic integration of transgenes into both alleles at any genomic locus and poor genomic stability due to heterozygosity at the allelic level. Use of a modified 3-isopropylmalate dehydrogenase (Leu2) cassette together with a gene of interest (GOI) cassette to development of a modified 3-isopropylmalate dehydrogenase (Leu2) S. boulardii unexpectedly addressed these issues with more robust expression levels, stability, and copy number of the desired gene of interest (i.e., therapeutic transgene).

The present disclosure provides for an engineered strain of S. boulardii yeast comprising a nucleic acid encoding Leu2 and at least one site-specific chromosomal insertion of a nucleic acid (i.e., a transgene) encoding a therapeutic protein.

In some embodiments, the engineered strain of S. boulardii yeast comprises one or more modifications (e.g., variant, mutation, deletion, insertion, etc.) that allows for selection of a particular engineered strain of S. boulardii (e.g., selectable marker). In some embodiments, the one or more modifications allows for selection comprising a partial deletion of a gene utilized as a selectable marker. In some embodiments, the one or more modifications that allows for selection comprises a complete deletion of a gene utilized as a selectable marker. In some embodiments, the one or more modifications reduce enzymatic activity of Leu2.

For example, the disclosed engineered S. boulardii yeast comprises the nucleic acid sequence encoding a modified Leu2 incorporated into the yeast genome. In some embodiments, the modified Leu2 comprises one or more modifications as compared to wild-type Leu2. In some embodiments, the nucleic acid encoding Leu2 is modified at the nicotinamide adenine dinucleotide (NAD+) binding region of Leu2. In some embodiments, the modification is a single point mutation or a base pair deletion. In some embodiments, the mutation is K90E. In some embodiments, the deletion can be any number of nucleotide base pair deletions. In some embodiments, the number of base pairs deleted can be between about 1 to about 40 base pairs. In some embodiments, the number of base pairs deleted can be between about 1 to about 5, between about 1 to about 10, between about 1 to about 15, between about 5 to about 10, between about 5 to about 15, between about 5 to about 20, between about 10 to about 15, between about 10 to about 20, between about 10 to about 25, between about 15 to about 20, between about 15 to about 25, between about 15 to 30, between about 20 to about 25, between about 20 to about 30, between about 20 to about 35, between about 25 to about 30, between about 25 to about 35, between about 25 to about 40, between about 30 to about 35, between about 30 to about 40, or between about 35 to about 40. In some embodiments, the deletion is 24 base pairs in length and the deletion comprises the nucleotide sequence of TTGGCCTCTTTGCCATCTGCGTCC (SEQ ID NO: 13). In some embodiments, the deletion is between positions 271-278 of the wild type Leu2 amino acid coding sequence. In some embodiments, the deletion comprises the amino acid sequence of LASLPSAS (SEQ ID NO: 14). In some embodiments, the Leu2 comprises an amino acid sequence selected from SEQ ID NOs 2 or 3. In some embodiments, the Leu2 comprises an amino acid sequence having at least 55%, at least 65%, at least 70%, at least 75%, at least 80%, at least 85%, at least 90%, at least 95%, at least 97%, at least 98%, at least 99%, or 100% sequence identity to SEQ ID NOs 2 or 3. In some embodiments, the Leu2 comprises an amino acid sequence of SEQ ID NO: 2. In some embodiments, the Leu2 comprises an amino acid sequence of SEQ ID NO: 3.

In some embodiments, the engineered strain of S. boulardii yeast as disclosed herein comprises a nucleic acid encoding a Leu2 promoter. The Leu2 promoter may be modified to weaken the promoter. In some embodiments, the Leu2 promoter comprises a nucleic acid sequence selected for SEQ ID NOs: 4 or 5. In some embodiments, the Leu2 promoter comprises a nucleic acid sequence selected form SEQ ID NO: 4. In some embodiments, the Leu2 promoter comprises a nucleic acid sequence selected form SEQ ID NO: 5.

Additionally, in some embodiments, the engineered strain of S. boulardii yeast as disclosed herein comprises a complete or partial deletion of wild-type Leu2. In some embodiments, the engineered strain of S. boulardii yeast comprises a complete or partial deletion of URA3. In some embodiments, the engineered strain of S. boulardii yeast comprises a complete or partial deletion of GAP1. In some embodiments, the engineered strain of S. boulardii yeast is ura3(−/−). In some embodiments, the engineered strain of S. boulardii yeast is gap1(−/−).

In some embodiments, the nucleic acid encoding the therapeutic transgene is incorporated into the yeast genome at one or more site specific chromosomal locations. In some embodiments, the nucleic acid encoding the therapeutic transgene is incorporated into at least 2 different chromosomes. In some embodiments, the nucleic acid encoding the therapeutic transgene is incorporated into at least 3, at least 4, at least 5, at least 6, at least 7, at least 8, at least 9, at least 10, at least 11, at least 12, at least 13, at least 14, at least 15, or at least 16 different chromosomes.

In some embodiments, the engineered strain of S. boulardii yeast as disclosed herein comprises at least 2, at least 3, at least 4, at least 5, at least 6, at least 7, at least 8, at least 9, or at least 10 or more copies of the nucleic acid encoding the therapeutic transgene incorporated into the yeast genome.

In some embodiments, the engineered strain of S. boulardii yeast is auxotrophic. In some embodiments, auxotrophic engineered strains of S. boulardii yeast are less likely to survive in the environment relative to an appropriate reference standard (e.g., a parental strain of S. boulardii yeast).

The engineered strain of S. boulardii can comprise a nucleic acid sequence encoding any therapeutic gene of interest (i.e., transgene) to express any therapeutic protein. In some embodiments, the engineered strain of S. boulardii expresses the therapeutic transgene. In some embodiments, the therapeutic protein is selected from a binding protein, an antigen-binding domain, an immunoglobulin, an antibody, a cytokine, a glycoprotein, an anti-cytokine, a hormone, a chemokine, an enzyme, an anti-microbial peptide, or any combination thereof.

In some embodiments, non-limiting examples of therapeutic proteins as disclosed encode for binding protein.

In some embodiments, non-limiting examples of therapeutic proteins as disclosed encode for a cytokine.

In some embodiments, the binding protein comprises a structure selected from VHH, Fc-VHH, VHH-Fc, VHH-VHH, VHH-VHH-VHH-VHH, Fc-VHH-VHH, VHH-Fc-VHH, and VHH-VHH-Fc, wherein each or any of the VHH or Fc domains are attached to another VHH or Fc domain via an optional linker sequence.

In some embodiments, the binding protein binds to Clostridioides difficile Toxin A (TcdA), Clostridioides difficile Toxin B (TcdB), or a combination of thereof. Referred to Antitoxin ABAB. In some embodiments, the therapeutic protein comprises the amino acid sequence of SEQ ID NO: 6.

In some embodiments, the binding protein binds to tumor necrosis factor alpha (TNF-α). In some embodiments, the binding protein is an IgG or comprises at least one VHH domain. In some embodiments, the therapeutic protein comprises an amino acid sequence selected from SEQ ID NOs 7 or 8.

In some embodiments, the binding protein binds to TNF-α and IL-17A. In some embodiments, the binding protein is an IgG or comprises at least two VHH domain. In some embodiments, the therapeutic protein comprises an amino acid sequence selected from of SEQ ID NOs 9-11.

In some embodiments, cytokine is human IL-10. In some embodiments, the IL-10 is Fc-fused or non Fc-fused. In some embodiments, the therapeutic protein comprises an amino acid sequence of SEQ ID NOs: 15.

In some embodiments, cytokine is human IL-22. In some embodiments, the IL-22 is Fc-fused or non Fc-fused. In some embodiments, the therapeutic protein comprises an amino acid sequence of SEQ ID NOs: 16.

In some embodiments, the cytokine is insulin. In some embodiments, the insulin is Fc-fused or non Fc-fused. In some embodiments, the therapeutic protein comprises an amino acid sequence of SEQ ID NOs: 17.

In some embodiments, the nucleic acid encoding the therapeutic protein as disclosed herein, comprises a linker (e.g., a peptide linker). In some embodiments, the linker has an amino acid sequence of GGGGSGGGGSGGGGS (SEQ ID NO: 12). Other linkers that may be used include, but are not limited to: repeats of glycine, repeats of glycine and serine, and repeats of alanine. In general, linkers that can connect one or more domains of a binding protein for the purposes of expression in an engineered yeast will comprise about 3 to about 20 amino acids. For example, a peptide linker may be 3, 4, 5, 6, 7, 8, 9, 10, 11, 12, 13, 14, 15, 16, 17, 18, 19, or 20 amino acids.

### III. Treatment or Prevention of Diseases and Conditions

The disclosed engineered strain of S. boulardii yeast can be used to treat any disease or condition by selecting a desirable therapeutic protein of interest to be expressed in the engineered strain of S. boulardii yeast that can target the disease or condition, thereby treating or preventing the disease or condition. The present disclosure provides for methods of treating or preventing a disease or condition, uses of the disclosed engineered strain of yeast, and/or the disclosed engineered yeast for use in treating a disease or condition, comprising orally administering to a subject in need thereof a therapeutically effective amount of the engineered strain of S. boulardii yeast as disclosed herein. The present disclosure additionally provides for methods of expressing a therapeutic protein of interest in a subject, comprising orally administering to the subject the engineered strain S. boulardii yeast as disclosed herein.

In some embodiments, non-limiting examples of diseases or conditions are selected from an inflammatory condition, an infection, irritable bowel syndrome (IBS), malignancy, neurodegenerative disease, diabetes, obesity, fatty liver disease, metabolic disease, graft-versus-host disease (GVHD), an autoimmune disease, or pain. In some embodiments, the inflammatory condition is selected from inflammatory bowel disease (IBD), gut inflammation, Crohn's disease, and ulcerative colitis. In some embodiments, the infection can be any infection, for example, an infection caused by C. difficile or a C. difficile infection.

A therapeutically effective amount can be administered in one or more administrations, applications, or dosages. Such delivery is dependent on a number of variables including, by not limited to, the time period for which the individual dosage unit is to be used and the bioavailability of the engineered strain of S. boulardii as disclosed herein. It is understood, however, that specific dose levels of the engineered stain of S. boulardii as disclosed herein for any particular subject depends upon a variety of factors including the activity of the specific compound employed, the age, body weight, general health, sex, and diet of the subject, the time of administration, the rate of excretion, the drug combination, and the severity of the particular disorder being treated and form of administration.

### IV. Pharmaceutical Compositions and Kits

The disclosed engineered strain of S. boulardii yeast can be provided in a pharmaceutical composition, such as a composition comprising the engineered strain of Saccharomyces yeast and a pharmaceutically acceptable carrier, excipient, and/or diluent. Non-limiting examples of suitable carriers, excipients, and diluents include lactose, dextrose, sucrose, trehalose, sorbitol, mannitol, xylitol, erythritol, maltitol, starch, acacia rubber, alginate, gelatin, calcium phosphate, calcium silicate, cellulose, methyl cellulose, microcrystalline cellulose, polyvinyl pyrrolidone, water, methylhydroxybenzoate, propylhydroxybenzoate, talc, magnesium stearate, minerals, and the like.

The pharmaceutical composition may be prepared for any route of administration, though in general, the pharmaceutical composition is suitable for oral administration.

In some embodiments, the engineered strain of S. boulardii yeast as disclosed herein is dissolved in water or another pharmaceutically acceptable aqueous carrier in which the conjugate exhibits good solubility, optionally with or without other pharmaceutical acceptable excipients, or preservatives.

Additionally, provided herein are kits. A kit may comprise one or more of the engineered strains of S. boulardii yeast as disclosed herein, contained in a suitable container, optionally together with instructions for use in a method as disclosed herein.

### V. Sequences

TABLE 1
List of Sequences.

| SEQ ID NO | Description | Sequence |
|---|---|---|
| 1 | Leu2 | MSAPKKIVVLPGDHVGQEITAEAIKVLKAISDVRSNVKFD |
| | | FENHLIGGAAIDATGVPLPDEALEASKKADAVLLGAVGG |
| | | PKWGTGSVRPKQGLLKIRKELQLYANLRPCNFASDSLLD |
| | | LSPIKPQFAKGTDFVVVRELVGGIYFGKRKEDDGDGVAW |
| | | DSEQYTVPEVQRITRMAAFMALQHEPPLPIWSLDKANVL |
| | | ASSRLWRKTVEETIKNEFPTLKVQHQLIDSAAMILVKNPT |
| | | HLNGIIITSNMFGDIISDEASVIPGSLGLLPSASLASLPSASL |
| | | ASLPDKNTAFGLYEPCHGSAPDLPKNKVNPIATILSAAM |
| | | MLKLSLNLPEEGKAIEDAVKKVLDAGIRTGDLGGSNSTT |
| | | EVGDAVAEEVKKILA |
| 2 | Modified Leu2 | MSAPKKIVVLPGDHVGQEITAEAIKVLKAISDVRSNVKFD |
| | | FENHLIGGAAIDATGVPLPDEALEASKKADAVLLGAVGG |
| | | PKWGTGSVRPEQGLLKIRKELQLYANLRPCNFASDSLLD |
| | | LSPIKPQFAKGTDFVVVRELVGGIYFGKRKEDDGDGVAW |
| | | DSEQYTVPEVQRITRMAAFMALQHEPPLPIWSLDKANVL |
| | | ASSRLWRKTVEETIKNEFPTLKVQHQLIDSAAMILVKNPT |
| | | HLNGIIITSNMFGDIISDEASVIPGSLGLLPSASLASLPSASL |
| | | ASLPDKNTAFGLYEPCHGSAPDLPKNKVNPIATILSAAM |
| | | MLKLSLNLPEEGKAIEDAVKKVLDAGIRTGDLGGSNSTT |
| | | EVGDAVAEEVKKILA |
| 3 | Modified Leu2 | MSAPKKIVVLPGDHVGQEITAEAIKVLKAISDVRSNVKFD |
| | | FENHLIGGAAIDATGVPLPDEALEASKKADAVLLGAVGG |
| | | PKWGTGSVRPKQGLLKIRKELQLYANLRPCNFASDSLLD |
| | | LSPIKPQFAKGTDFVVVRELVGGIYFGKRKEDDGDGVAW |
| | | DSEQYTVPEVQRITRMAAFMALQHEPPLPIWSLDKANVL |
| | | ASSRLWRKTVEETIKNEFPTLKVQHQLIDSAAMILVKNPT |
| | | HLNGIIITSNMFGDIISDEASVIPGSLGLLPSASLASLPDKNT |
| | | AFGLYEPCHGSAPDLPKNKVNPIATILSAAMMLKLSLNLP |
| | | EEGKAIEDAVKKVLDAGIRTGDLGGSNSTTEVGDAVAEE |
| | | VKKILA |
| 4 | Leu2 promoter (1) | GTGGTTAGCAATCGTCTTACTTTCTAACTTTTCTTACCTTTTA |
| | | CATTTCAGCAATATATATATATATATATTTCAAGGATATACC |
| | | ATTGTA |
| 5 | Leu2 promoter (2) | TATATATATATTTCAAGGATATACCATTGTA |
| 6 | Exemplary | QVQLVETGGGLVQPGGSLRLSCAASGFTLDYSSIGWFRQAPGK |
| | antitoxin ABAB | EREGVSCISSSGDSTKYADSVKGRFTTSRDNAKNTVYLQMNSL |
| | sequence | KPDDTAVYYCAAFRATMCGVFPLSPYGKDDWGKGTLVTVSSG |
| | | GGGSGGGGSGGGGSQVQLVESGGGLVQPGGSLRLSCEASGFTL |
| | | DYYGIGWFRQPPGKEREAVSYISASARTILYADSVKGRFTISRD |
| | | NAKNAVYLQMNSLKREDTAVYYCARRRFSASSVNRWLADDY |
| | | DVWGRGTQVAVSSGGGSGGGSGGGSGGGSQLQLVETGGGLV |
| | | QPGGSLRLSCAASGFTFSDYVMTWVRQAPGKGPEWIATINTDG |
| | | STMRDDSTKGRFTISRDNAKNTLYLQMTSLKPEDTALYYCARG |
| | | RVISASAIRGAVRGPGTQVTVSSGGGGSGGGGSGGGGSQVQLV |
| | | ESGGGLVQTGGSLRLSCASSGSIAGFETVTWSRQAPGKSLQWV |
| | | ASMTKTNNEIYSDSVKGRFIISRDNAKNTVYLQMNSLKPEDTG |
| | | VYFCKGPELRGQGIQVTVSS |
| 7 | Exemplary anti- | EVQLLESGGGLVQPGGSLRLSCAASGRTFSTYAMGWFRQAPG |
| | TNF-α (human) | KEREFVATVDNDGFTMHADSVKGRFTISRDNSKNTLYLQMNS |
| | | LRAEDTAVYYCAAGQYSKFVESYAYWGQGTLVTVSS |
| 8 | Exemplary anti- | QVQLQDSGGGLVQAGGSLRLSCAASGGTFSSIIMAWFRQAPGK |
| | TNF-α (mouse) | EREFVGAVSWSGGTTVYADSVLGRFEISRDSARKSVYLQMNSL |
| | | KPEDTAVYYCAARPYQKYNWASASYNVWGQGTQVTVSSGGG |
| | | GSGGGGSGGGGSQVQLQDSGGGLVQAGGSLRLSCAASGGTFS |
| | | SIIMAWFRQAPGKEREFVGAVSWSGGTTVYADSVLGRFEISRD |
| | | SARKSVYLQMNSLKPEDTAVYYCAARPYQKYNWASASYNVW |
| | | GQGTQVTVSS |
| 9 | Exemplary bi- | EVQLLESGGGLVQPGGSLRLSCAASGRTFSTYAMGWFRQAPG |
| | specific antibody | KEREFVATVDNDGFTMHADSVKGRFTISRDNSKNTLYLQMNS |
| | against IL-17A | LRAEDTAVYYCAAGQYSKFVESYAYWGQGTLVTVSSGQAGQ |
| | and TNF-α | GGGGSGGGGSGGGGSLQQVQLVQSGGGLVQAGGSLRLSCAAS |
| | | GGTFATSPMGWLRQAPGKGTEFVAAISPSGGDRIYADSVKGRF |
| | | TISRDNAGYFIYLQMNSLKPEDTAVYYCAVRRRFDGTSYYTGD |
| | | YDSWGQGTLVTVSS |
| 10 | VHH1 (anti-TNF-α) | EVQLLESGGGLVQPGGSLRLSCAASGRTFSTYAMGWFRQAPG |
| | | KEREFVATVDNDGFTMHADSVKGRFTISRDNSKNTLYLQMNS |
| | | LRAEDTAVYYCAAGQYSKFVESYAYWGQGTLVTVSSGQAGP |
| 11 | VHH2 (anti-IL- | QVQLVQSGGGLVQAGGSLRLSCAASGGTFATSPMGWLRQAPG |
| | 17A) | KGTEFVAAISPSGGDRIYADSVKGRFTISRDNAGYFIYLQMNSL |
| | | KPEDTAVYYCAVRRRFDGTSYYTGDYDSWGQGTLVTVSS |
| 12 | Exemplary Linker | GGGGSGGGGSGGGGS |
| 13 | Leu2 deletion | TTGGCCTCTTTGCCATCTGCGTCC |
| 14 | Leu2 deletion | LASLPSAS |
| 15 | Exemplary IL-10 | SPGQGTQSENSCTHFPGNLPNMLRDLRDAFSRVKTFFQMKDQL |
| | (human) | DNLLLKESLLEDFKGYLGCQALSEMIQFYLEEVMPQAENQDPD |
| | | IKAHVNSLGENLKTLRLRLRRCHRFLPCENKSKAVEQVKNAFN |
| | | KLQEKGIYKAMSEFDIFINYIEAYMTMKIRN |
| 16 | Exemplary IL-22 | APISSHCRLDKSNFQQPYITNRTFMLAKEASLADNNTDVRLIGE |
| | (human) | KLFHGVSMSERCYLMKQVLNFTLEEVLFPQSDRFQPYMQEVVP |
| | | FLARLSNRLSTCHIEGDDLHIQRNVQKLKDTVKKLGESGEIKAI |
| | | GELDLLFMSLRNACI |
| 17 | Exemplary Insulin | FVNQHLCGSHLVEALELVCGERGFHYGGGGGGSGGGGGIVEQ |
| | (human) | CCTSTCSLDQLENYCG |

### EXAMPLES

These examples are provided for illustrative purposes only and not to limit the scope of the claims provided herein.

### Example 1. Development of a Strain of Saccharomyces boulardii Yeast for Biologic Therapies

Preparation of S. boulardii Ura3(−/−), Gap1(−/−), Leu2(−/−) Background Strain

The probiotic yeast Saccharomyces boulardii (S. boulardii) was engineered to utilize auxotrophic markers to facilitate multi-copies of a gene of interest (GOI) for the development of therapeutic probiotic yeast strains. A double knockout of URA3 and GAP1 version of MYA-796, a parental diploid strain, from ATCC (Saccharomyces cerevisiae Meyen ex. E. C. Hansen (also known as Saccharomyces boulardii or Saccharomyces cerevisiae var. boulardii); GenBank JRHY00000000) was utilized as a background strain. The nuclear sequence spanning 75592 to 77147 containing Leu2 of chromosome III was targeted using CRISPR/Cas9 to create a double knockout of Leu2.

Optimization of GOI Cassette with Modified Leu2 Compared to DHFR

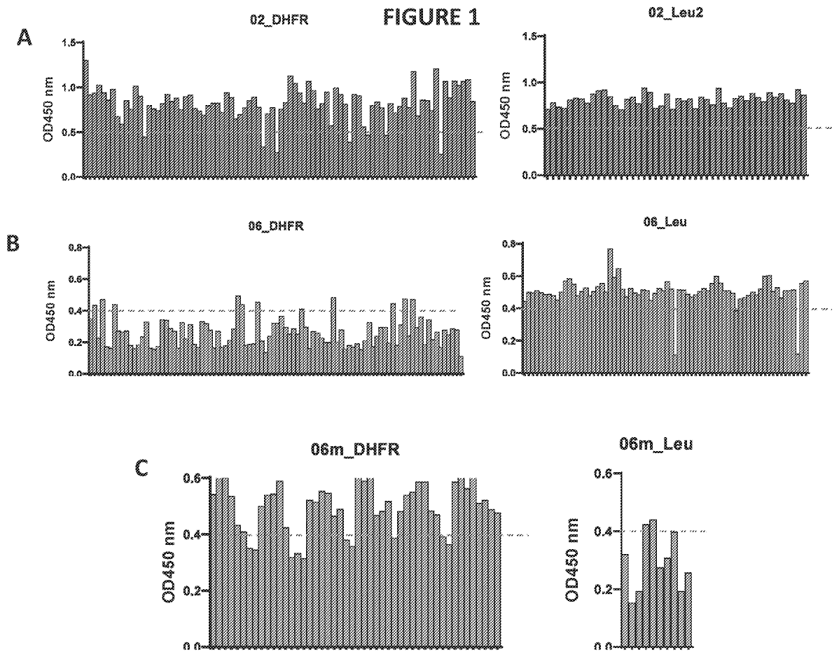

A modified Leu2 gene was utilized to prepare a GOI cassette with Leu2 selection. Specifically, the NAD+ binding regions of the Leu2 gene was targeted with either a K90E mutation or a 24 bp deletion (SEQ ID NO:13) in the NAD+ binding site using Leu2 promoters selected from SEQ ID NOs: 4 or 5. The background strain was transformed with the GOI insertion cassettes individually, comprising the K90E mutation modified version of the Leu2 gene. Additionally, the background strain was transformed with a GOI insertion cassette comprising DHFR selection marker. The transformants were individually picked and seeded in YPD for overnight culture (shaking) to induce therapeutic protein expression from Leu2 and DHFR selection clones. Culture supernatants were diluted 40-fold for DHFR selection and Leu2 selection for expression measurement of a GOI by ELISA (FIG. 1A), diluted 25-fold for DHFR selection and 40-fold for Leu2 selection for expression measurement of a GOI by ELISA (FIG. 1B), or diluted 5-fold for DHFR selection and 10-fold for Leu2 selection for expression measurement of a GOI by ELISA (FIG. 1C).

### Example 2: Comparison of Exemplary Protein Copy Number Using Leu2 Selection Compared to DHFR Selection

The background strain as described in Example 1 was transformed with the GOI cassette consisting of exemplary DNA sequences encoding an antibody against cytokine, therapeutic protein, or tetra-specific antibody-encoding transgene of interest and the K90E mutation modified version of the Leu2 gene or the DHFR gene. The transformants were individually picked and seeded in YPD for overnight culture (shaking) to induced expression and the copy number of the cytokine, therapeutic protein or tetra-specific antibody was determined. Results demonstrated that Leu2 selection produces similar level of copy number as with DHFR selection. (Table 2).

TABLE 2
Copy number of exemplary therapeutic strain development

Copy Number

| Protein Expressed | Leu2 Selection | DHFR Selection |
|---|---|---|
| Anti-Cytokine | 11 | 12 |
| Therapeutic Protein | 11 | 7 |
| Tetra-Specific Antibody | 10 | 9 |

### Example 3: Comparison of Exemplary Protein Expression Using Leu2 Selection Compared to DHFR Selection at 3 Hour Acute Expression and 24 Hour Expression

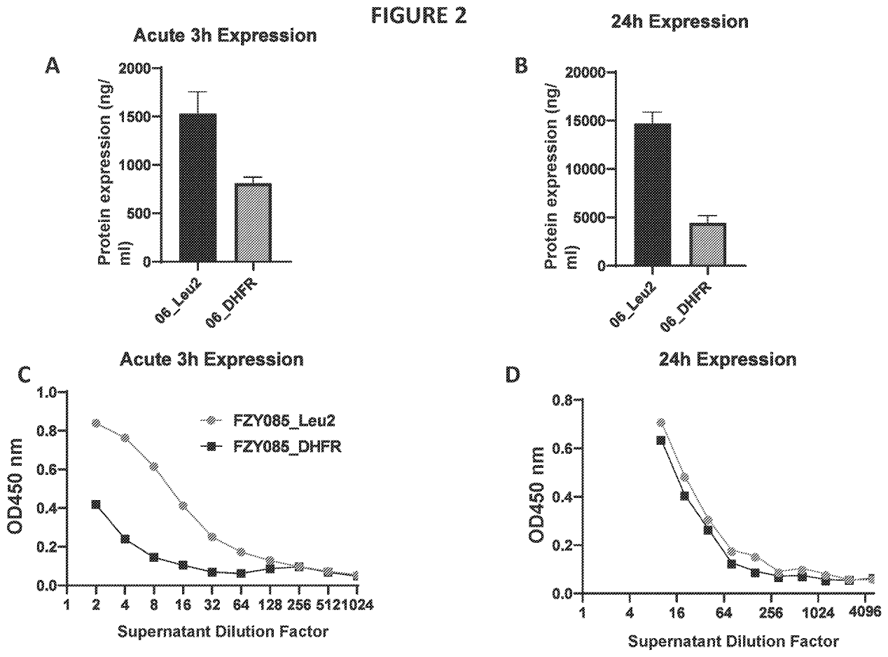

The background strain as described in Example 1 was transformed with the GOI cassette consisting of an exemplary DNA sequence encoding an antibody and the K90E mutation modified version of the Leu2 gene or the DHFR gene. The engineered yeast cells were initially cultured in minimal selection (M1) medium for overnight, then seeded in YPD at an OD600 of 0.2 for 24 hour expression, and an OD600 of 3 for 3 hour acute expression. Cultures were incubated with shaking at 250 rpm at 30° C. for 24 hour expression and at 37° C. for the 3 hour acute expression. Cultured supernatants were collected and antibody (anti-TNF-α (human)) was measured by ELISA at 3 hour acute expression (FIG. 2A) and 24 hour expression (FIG. 2B). Additionally, for the 3 hour acute expression, supernatants were diluted by a dilution factor of 2, 4, 8, 16, 32, 64, 128, 256, 512, and 1024, and the antibody (anti-TNF-α (mouse)) levels were measured by ELISA (FIG. 2C). For the 24 hour expression of anti-TNF-α (mouse), the supernatants were diluted by a dilution factor of 10, 20, 40, 80, 160, 320, 640, 1280, 2560, and 5120, and the cytokine protein levels were measured by ELISA (FIG. 2D). Leu2 strains had a much higher expression or similar expression of the cytokine compared to DHFR strains.

### Example 4. Evaluation of Leu2 Strain Expression Over Varying Time Points of Culture Conditions

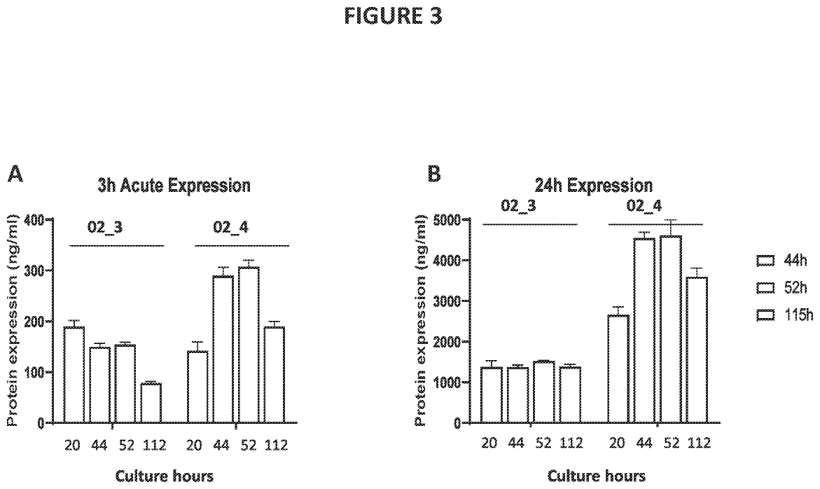

The background strain as described in Example 1 was transformed with the GOI cassette consisting of antitoxin ABAB and the K90E mutation modified version of the Leu2 gene. The engineered yeast cells (Leu2 stains 02_3 and 02_4) were initially cultured in M1 medium, then seeded at an OD600 of 0.2 in M2 and cultured for 20, 44, 52, and 112 hours. At the different time points, yeast cells were harvested and resuspended in YPD to prepare samples for 3 hour acute expression and 24-hour expression measurements. For 3 hour acute expression samples, engineered yeast cells were resuspended in 1 ml of YPD at a density of OD600 of 9. For 24-hour expression, engineered yeast cells were resuspended in 0.2 ml of YPD at a density of OD600 of 3. Prepared samples were then frozen at −80° C. for at least 18 hours. Subsequently, samples were thawed at room temperature (RT) and recovered to a volume of 3 ml with YPD (adjusted to a density of OD600 of 3 for 3 hour acute expression and OD600 of 0.2 for 24-hour expression) before being cultured at 37° C. with shaking at 250 rpm. Quantitative ELISA was conducted, and expression levels were analyzed using standards as references. (FIG. 3A-B).

### Example 5. Evaluation of Leu2 Strain Stability

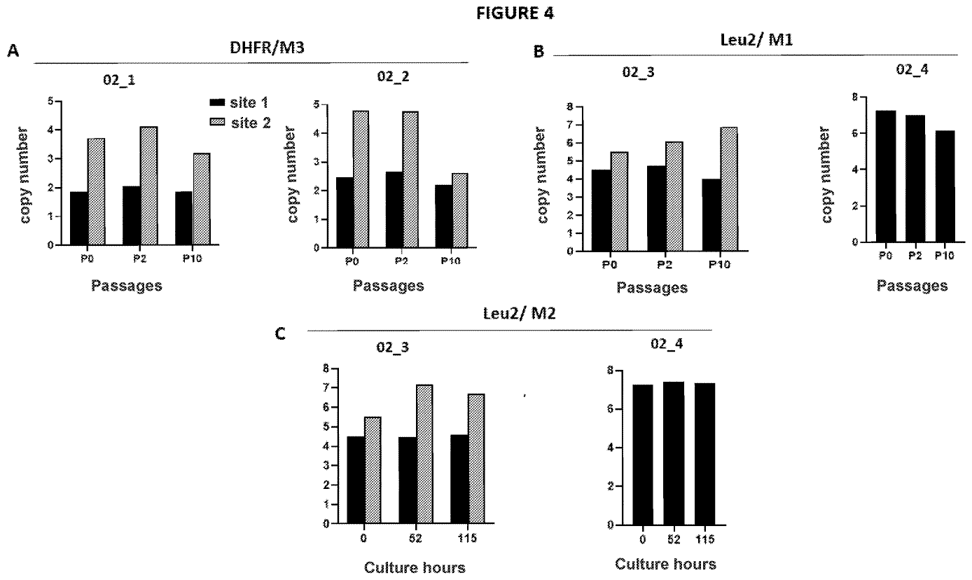

The background strain as described in Example 1 was transformed with the GOI cassette consisting of antitoxin ABAB and the K90E mutation modified version of the Leu2 gene or the DHFR gene. The engineered yeast cells were cultured in M1 medium for the Leu2 strains and M3 medium for the DHFR strains, with shaking at 250 rpm at 30° C. for daily passage at a seeding density of OD600 of 0.2 for 10 days, corresponding to approximately 120 generations of therapeutic strains. Cells were harvested for genomic DNA extraction at passage 0, 2, and 10, and qPCR was performed to measure the copy number of small fragments (around 200 bp) in GOI cassette at two different sites of integration. The Leu2 strains produce a higher copy number compared to the DHFR strains (FIG. 4A-B). Cells of Leu2 strains were also harvested from M2 medium culture for genomic DNA extraction at culture hours 0, 52, and 115 for Leu2 strains. (FIG. 4C). Copy number is kept relatively more stable in Leu2 strains.

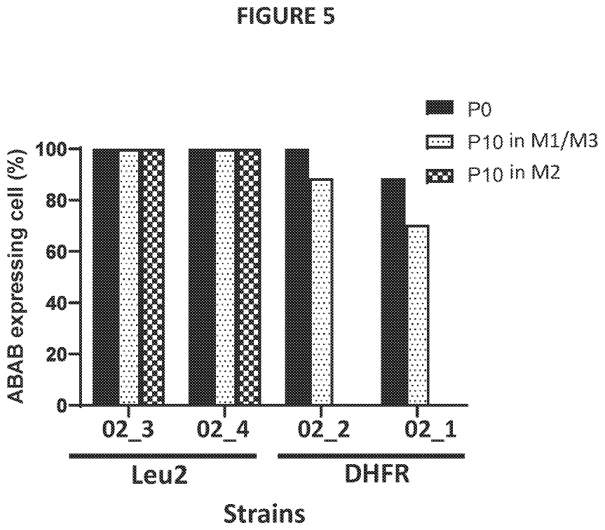

Additionally, the engineered yeast cells were seeded at an OD600 of 0.2 in M1 and M2 medium for Leu2 strains, and M3 medium for DHFR strains, and cultured for 24 hours per passage, continuing for 10 passages at 30° C. with shaking at 250 rpm. Cultured cells were spread on corresponding plate at day 0 (P0) and day 10 (P10) and single colonies grown on these plate were randomly selected and seeded into YPD medium in a 96-well culture plate for an overnight shaking culture. The therapeutic protein expression was detected by ELISA using the culture supernatant from each single colony. Clones with positive expression of therapeutic protein are identified as specific protein-expressing cells. The Leu2 maintained 100 percent of ABAB expressing cells, whereas the DHFR strains result in a decrease in the percentage of ABAB expressing cells at passage 10 (FIG. 5).

### Example 6. Evaluation of Leu2 Strain Growth

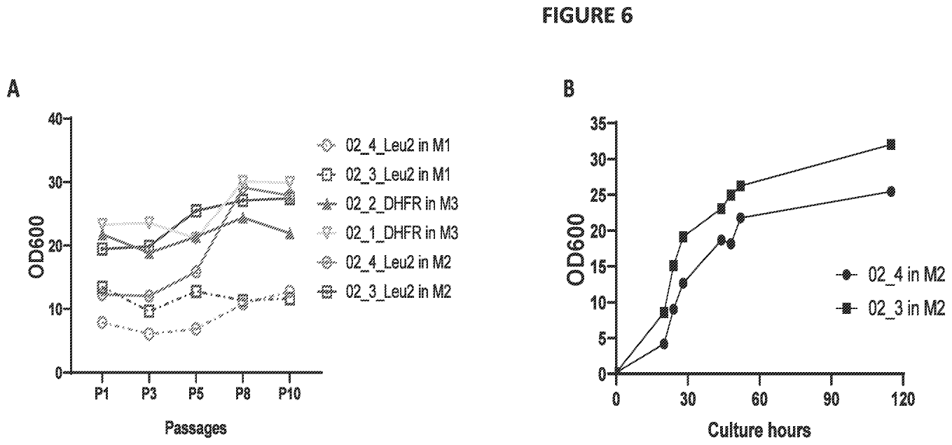

The background strain as described in Example 1 was transformed with the GOI cassette consisting of antitoxin ABAB and the K90E mutation modified version of the Leu2 gene or the DHFR gene. The engineered yeast cells were seeded at an OD600 of 0.2 in M1 and M2 medium for Leu2 strains (02_4 and 02_3), and M3 medium for DHFR strains (02_2 and 02_1), and cultured for 24 hours per passage, continuing for 10 passages at 30° C. with shaking at 250 rpm. OD600 was measured for each passage (passage 1, 3, 5, 8, and 10) to evaluate the growth of the engineered yeast cells. The growth of Leu2 strains in M1 is robust, in M2 medium have comparable growth to DHFR strains (FIG. 6A).

Additionally, OD600 was continuously measured for the Leu2 strains cultured in M2 medium for 0 hours, 20 hours, 24 hours, 28 hours, 44 hours, 48 hours, 52 hours, and 115 hours demonstrating increased growth patterns as the hours of culture increases (FIG. 6B).

### Example 7. Evaluation of Leu2 Strain Expression

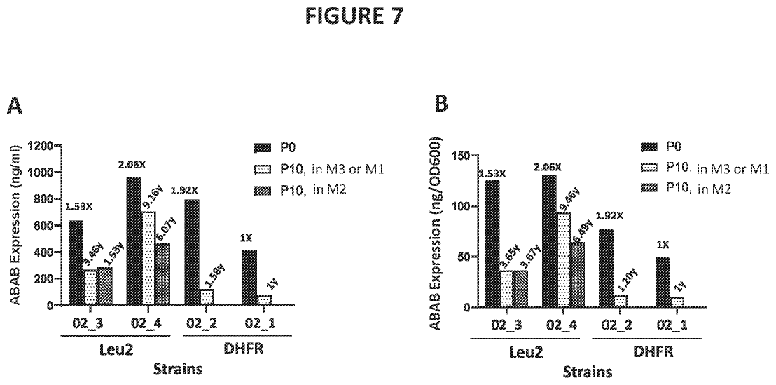

The background strain as described in Example 1 was transformed with the GOI cassette consisting of antitoxin ABAB and the K90E mutation modified version of the Leu2 gene or the DHFR gene. The engineered yeast cells were seeded at an OD600 of 0.2 in M1 and M2 medium for Leu2 strains (02_3 and 02_4), and −M3 medium for DHFR strains (02_2 and 02_1), and cultured for 24 hours per passage, continuing for 10 passages at 30° C. with shaking at 250 rpm. Cultured cells from passages 0 (P0) and 10 (P10) were then seeded in YPD at an OD600 of 0.2 and cultured as in the passage medium for 24 hours. Culture supernatants were assayed for expression of ABAB by ELISA (FIG. 7 left). OD600 was measured at the end of growth in YPD to calculate the expression/OD600 (FIG. 7 right). The Leu2 strains present increased ABAB expression and increased growth at P10 compared to DHFR strains.

### Example 8. Potent Containment Evaluation of Leu2 Strains

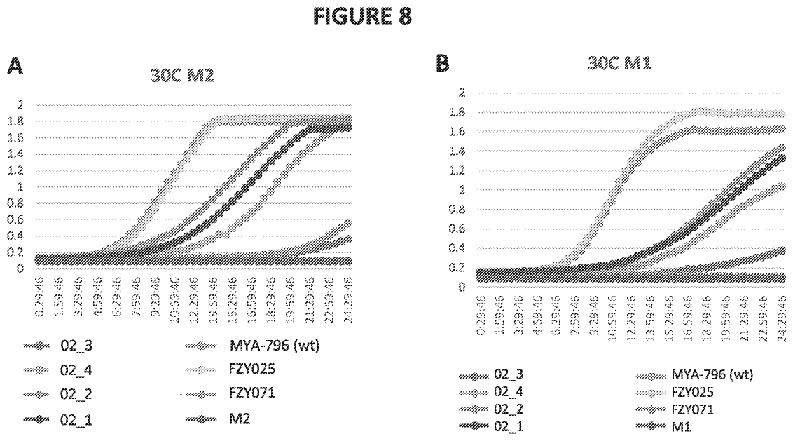

The background strain as described in Example 1 was transformed with the GOI cassette consisting of antitoxin ABAB and the K90E mutation modified version of the Leu2 gene or the DHFR gene. The engineered yeast cells for Leu2 strains (02_4 and 02_3) and for DHFR strains (02_2 and 02_1), as well as related parental strains from which derived the above Leu2 and DHFR strains (such as MYA-796, FZY025, FZY071) were cultured in 1 ml of M1 or M2 medium individually with a seeding density of 0.1 OD600 in a 24-well plate for overnight at 30 C with shaking at 250 rpm. Y axis represents OD600 (cell density indicates the growth of yeast cells) and x axis is time points, at which the growth (OD600) is recorded. Based on the growth shift to the far right of Leu2 strains compared to DHFR strains and related parental strains either in selection medium (M1) (FIG. 8A) and nutrient-limited medium (M2) (FIG. 8B), it indicates that slow/arrest-like growth of Leu2 strains are potent containment capability and less adverse effect on environment.

These examples are provided for illustrative purposes only and not to limit the scope of the claims provided herein. While certain embodiments have been illustrated and described, it should be understood that changes and modifications can be made therein in accordance with ordinary skill in the art without departing from the technology in its broader aspects as defined in the following claims.

The present disclosure is not to be limited in terms of the particular embodiments described in this application. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, or compositions, which can of course vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

1. An engineered strain of Saccharomyces boulardii yeast comprising a nucleic acid encoding 3-isopropylmalate dehydrogenase (Leu2) and at least one site-specific chromosomal insertion of a nucleic acid encoding a therapeutic protein.

2-3. (canceled)

4. The engineered strain of Saccharomyces boulardii yeast of claim 1, wherein the Leu2 comprises an amino acid sequence selected from SEQ ID NOs: 2 or 3.

5. (canceled)

6. The engineered strain of Saccharomyces boulardii yeast of claim 1, further comprising a complete or partial deletion of URA3.

7. The engineered strain of Saccharomyces boulardii yeast of claim 1, further comprising a complete or partial deletion of GAP1.

8. (canceled)

9. The engineered strain of Saccharomyces boulardii yeast of claim 1, wherein the nucleic acid encoding the therapeutic protein is incorporated into at least two different chromosomes.

10. (canceled)

11. The engineered strain of Saccharomyces boulardii yeast of claim 1, further comprising a Leu2 promoter, wherein the Leu2 promoter comprises a nucleic acid sequence selected from SEQ ID NOs: 4 or 5.

12. (canceled)

13. The engineered strain of Saccharomyces boulardii yeast of claim 1, wherein the therapeutic protein is a binding protein that binds to TcdA, TcdB, or a combination thereof.

14. The engineered strain of Saccharomyces boulardii yeast of claim 13, wherein the therapeutic protein comprises an amino acid sequence of SEQ ID NO: 6.

15. The engineered strain of Saccharomyces boulardii yeast of claim 1, wherein the therapeutic protein is a binding protein that is an IgG or comprises at least one VHH domain that binds to TNF-α.

16. (canceled)

17. The engineered strain of Saccharomyces boulardii yeast of claim 15, wherein the therapeutic protein comprises an amino acid sequence of SEQ ID NOs: 7 or 8.

18. The engineered strain of Saccharomyces boulardii yeast of claim 1, wherein the therapeutic protein is a binding protein that is an IgG or comprises at least two VHH domains and binds to TNF-α and IL-17A.

19. (canceled)

20. The engineered strain of Saccharomyces boulardii yeast of claim 18, wherein the therapeutic protein comprises an amino acid sequence of SEQ ID NOs: 9-11.

21. The engineered strain of Saccharomyces boulardii yeast of claim 1, wherein the therapeutic protein expresses human cytokine IL-10 that is Fc-fused or non Fc-fused.

22. (canceled)

23. The engineered strain of Saccharomyces boulardii yeast of claim 21, wherein the therapeutic protein comprises an amino acid sequence of SEQ ID NOs: 15.

24. The engineered strain of Saccharomyces boulardii yeast of claim 1, wherein the therapeutic protein expresses human cytokine IL-22 that is Fc-fused or non Fc-fused.

25. (canceled)

26. The engineered strain of Saccharomyces boulardii yeast of claim 24, wherein the therapeutic protein comprises an amino acid sequence of SEQ ID NOs: 16.

27. The engineered strain of Saccharomyces boulardii yeast of claim 1, wherein the therapeutic protein expresses human cytokine insulin that is Fc-fused or non Fc-fused.

28. (canceled)

29. The engineered strain of Saccharomyces boulardii yeast of claim 27, wherein the therapeutic protein comprises an amino acid sequence of SEQ ID NOs: 17.

30. (canceled)

31. A method of treating or preventing a disease or condition comprising orally administering to a subject in need thereof a therapeutically effective amount of the engineered strain of Saccharomyces boulardii yeast of claim 1, wherein the disease or condition is selected from an inflammatory condition, an infection, irritable bowel syndrome (IBS), malignancy, neurodegenerative disease, diabetes, obesity, fatty liver disease, metabolic disease, graft-versus-host disease (GVHD), an autoimmune disease, or pain.

32-35. (canceled)

36. A pharmaceutical composition comprising the engineered strain of Saccharomyces boulardii yeast of claim 1 and a pharmaceutically acceptable carrier or diluent.

37-38. (canceled)


SEQUENCE LISTING (17 sequences)

SEQ ID NO: 1 - protein, 372 aa, synthetic construct
MSAPKKIVVLPGDHVGQEITAEAIKVLKAISDVRSNVKFDFENHLIGGAAIDATGVPLPDEALEASKKADAVLLGAVGGPKWGTGSVRPKQGLLKIRKELQLYANLRPCNFASDSLLDLSPIKPQFAKGTDFVVVRELVGGIYFGKRKEDDGDGVAWDSEQYTVPEVQRITRMAAFMALQHEPPLPIWSLDKANVLASSRLWRKTVEETIKNEFPTLKVQHQLIDSAAMILVKNPTHLNGIIITSNMFGDIISDEASVIPGSLGLLPSASLASLPSASLASLPDKNTAFGLYEPCHGSAPDLPKNKVNPIATILSAAMMLKLSLNLPEEGKAIEDAVKKVLDAGIRTGDLGGSNSTTEVGDAVAEEVKKILA

SEQ ID NO: 2 - protein, 372 aa, synthetic construct
MSAPKKIVVLPGDHVGQEITAEAIKVLKAISDVRSNVKFDFENHLIGGAAIDATGVPLPDEALEASKKADAVLLGAVGGPKWGTGSVRPEQGLLKIRKELQLYANLRPCNFASDSLLDLSPIKPQFAKGTDFVVVRELVGGIYFGKRKEDDGDGVAWDSEQYTVPEVQRITRMAAFMALQHEPPLPIWSLDKANVLASSRLWRKTVEETIKNEFPTLKVQHQLIDSAAMILVKNPTHLNGIIITSNMFGDIISDEASVIPGSLGLLPSASLASLPSASLASLPDKNTAFGLYEPCHGSAPDLPKNKVNPIATILSAAMMLKLSLNLPEEGKAIEDAVKKVLDAGIRTGDLGGSNSTTEVGDAVAEEVKKILA

SEQ ID NO: 3 - protein, 364 aa, synthetic construct
MSAPKKIVVLPGDHVGQEITAEAIKVLKAISDVRSNVKFDFENHLIGGAAIDATGVPLPDEALEASKKADAVLLGAVGGPKWGTGSVRPKQGLLKIRKELQLYANLRPCNFASDSLLDLSPIKPQFAKGTDFVVVRELVGGIYFGKRKEDDGDGVAWDSEQYTVPEVQRITRMAAFMALQHEPPLPIWSLDKANVLASSRLWRKTVEETIKNEFPTLKVQHQLIDSAAMILVKNPTHLNGIIITSNMFGDIISDEASVIPGSLGLLPSASLASLPDKNTAFGLYEPCHGSAPDLPKNKVNPIATILSAAMMLKLSLNLPEEGKAIEDAVKKVLDAGIRTGDLGGSNSTTEVGDAVAEEVKKILA

SEQ ID NO: 4 - DNA, 91 bp, synthetic construct
  source 1..91: mol_type = other DNA
gtggttagcaatcgtcttactttctaacttttcttaccttttacatttcagcaatatatatatatatatatttcaaggatataccattgta

SEQ ID NO: 5 - DNA, 31 bp, synthetic construct
  source 1..31: mol_type = other DNA
tatatatatatttcaaggatataccattgta

SEQ ID NO: 6 - protein, 532 aa, synthetic construct
QVQLVETGGGLVQPGGSLRLSCAASGFTLDYSSIGWFRQAPGKEREGVSCISSSGDSTKYADSVKGRFTTSRDNAKNTVYLQMNSLKPDDTAVYYCAAFRATMCGVFPLSPYGKDDWGKGTLVTVSSGGGGSGGGGSGGGGSQVQLVESGGGLVQPGGSLRLSCEASGFTLDYYGIGWFRQPPGKEREAVSYISASARTILYADSVKGRFTISRDNAKNAVYLQMNSLKREDTAVYYCARRRFSASSVNRWLADDYDVWGRGTQVAVSSGGGSGGGSGGGSGGGSQLQLVETGGGLVQPGGSLRLSCAASGFTFSDYVMTWVRQAPGKGPEWIATINTDGSTMRDDSTKGRFTISRDNAKNTLYLQMTSLKPEDTALYYCARGRVISASAIRGAVRGPGTQVTVSSGGGGSGGGGSGGGGSQVQLVESGGGLVQTGGSLRLSCASSGSIAGFETVTWSRQAPGKSLQWVASMTKTNNEIYSDSVKGRFIISRDNAKNTVYLQMNSLKPEDTGVYFCKGPELRGQGIQVTVSS

SEQ ID NO: 7 - protein, 120 aa, synthetic construct
EVQLLESGGGLVQPGGSLRLSCAASGRTFSTYAMGWFRQAPGKEREFVATVDNDGFTMHADSVKGRFTISRDNSKNTLYLQMNSLRAEDTAVYYCAAGQYSKFVESYAYWGQGTLVTVSS

SEQ ID NO: 8 - protein, 263 aa, synthetic construct
QVQLQDSGGGLVQAGGSLRLSCAASGGTFSSIIMAWFRQAPGKEREFVGAVSWSGGTTVYADSVLGRFEISRDSARKSVYLQMNSLKPEDTAVYYCAARPYQKYNWASASYNVWGQGTQVTVSSGGGGSGGGGSGGGGSQVQLQDSGGGLVQAGGSLRLSCAASGGTFSSIIMAWFRQAPGKEREFVGAVSWSGGTTVYADSVLGRFEISRDSARKSVYLQMNSLKPEDTAVYYCAARPYQKYNWASASYNVWGQGTQVTVSS

SEQ ID NO: 9 - protein, 267 aa, synthetic construct
EVQLLESGGGLVQPGGSLRLSCAASGRTFSTYAMGWFRQAPGKEREFVATVDNDGFTMHADSVKGRFTISRDNSKNTLYLQMNSLRAEDTAVYYCAAGQYSKFVESYAYWGQGTLVTVSSGQAGQGGGGSGGGGSGGGGSLQQVQLVQSGGGLVQAGGSLRLSCAASGGTFATSPMGWLRQAPGKGTEFVAAISPSGGDRIYADSVKGRFTISRDNAGYFIYLQMNSLKPEDTAVYYCAVRRRFDGTSYYTGDYDSWGQGTLVTVSS

SEQ ID NO: 10 - protein, 125 aa, synthetic construct
EVQLLESGGGLVQPGGSLRLSCAASGRTFSTYAMGWFRQAPGKEREFVATVDNDGFTMHADSVKGRFTISRDNSKNTLYLQMNSLRAEDTAVYYCAAGQYSKFVESYAYWGQGTLVTVSSGQAGP

SEQ ID NO: 11 - protein, 125 aa, synthetic construct
QVQLVQSGGGLVQAGGSLRLSCAASGGTFATSPMGWLRQAPGKGTEFVAAISPSGGDRIYADSVKGRFTISRDNAGYFIYLQMNSLKPEDTAVYYCAVRRRFDGTSYYTGDYDSWGQGTLVTVSS

SEQ ID NO: 12 - protein, 15 aa, synthetic construct
GGGGSGGGGSGGGGS

SEQ ID NO: 13 - DNA, 24 bp, synthetic construct
  source 1..24: mol_type = other DNA
ttggcctctttgccatctgcgtcc

SEQ ID NO: 14 - protein, 8 aa, synthetic construct
LASLPSAS

SEQ ID NO: 15 - protein, 160 aa, synthetic construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 16 - protein, 146 aa, synthetic construct
APISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACI

SEQ ID NO: 17 - protein, 58 aa, synthetic construct
FVNQHLCGSHLVEALELVCGERGFHYGGGGGGSGGGGGIVEQCCTSTCSLDQLENYCG